(19) United States
(12) Patent Application Publication
(10) Pub. No.: US 2014/0242628 A1
(43) Pub. Date: Aug. 28, 2014

(54) ANALYTICAL METHOD AND DEVICE FOR MALIC ACID

(71) Applicant: Sportsman Consulting, LLC, Carlsbad, CA (US)

(72) Inventors: John Richard Sportsman, Encinitas, CA (US); Richard William Sportsman, Encinitas, CA (US)

(73) Assignee: Sportsman Consulting, LLC, Carlsbad, CA (US)

(21) Appl. No.: 14/188,919

(22) Filed: Feb. 25, 2014

Related U.S. Application Data
(60) Provisional application No. 61/769,071, filed on Feb. 25, 2013.
(60) Provisional application No. 61/938,561, filed on Feb. 11, 2014.

(51) Int. Cl. C12Q 1/26; G01N 33/14

(52) U.S. Cl. CPC C12Q 1/26; G01N 33/146
USPC 435/25

(57) ABSTRACT

Analytical methods and devices according to the present disclosure may be used to assay the concentration of malic acid, or related species, in a sample, e.g., preparations of fruit, vegetables, juice, and/or wine. Liquid samples are combined with a reaction mixture, incubated, and a parameter characteristic of the resulting incubated mixture is measured. During the incubation, the reaction mixture generally reacts with the liquid sample to transform malic acid or related species that is present in the liquid sample. The measured parameter may be pressure, pH, an amount (e.g., volume, mass) of CO₂ generated during the incubation, a parameter related to the foregoing, and/or any other parameter related to the amount of malic acid or related species transformed.

### RELATED MATERIALS

This application claims priority under 35 U.S.C. §119(e) to U.S. Provisional Patent Application No. 61/769,071, which was filed Feb. 25, 2013, and to U.S. Provisional Patent Application No. 61/938,561, which was filed Feb. 11, 2014, the complete disclosures of which are incorporated herein by reference for all purposes. This application incorporates by reference in their entirety for all purposes all patent and non-patent references cited anywhere in this application.

### FIELD

The present disclosure relates to analytical methods and devices for malic acid.

### BACKGROUND

Malic acid (also referred to as I-malic acid, I-malate, and (S)-malate) is naturally present in many fruits, vegetables, juices, and grape musts (from which wine is fermented). Frequently, it is desirable to convert the malic acid (e.g., in the grape must or wine) into lactic acid by a secondary fermentation known as malolactic fermentation.

Winemakers typically use live organisms, e.g., malolactic bacteria Oenococcus oeni, to perform malolactic fermentation in wine. Before adding O. oeni or similar organisms, inhibitory compounds, such as sulfite, are reduced in, or removed from, the grape must or wine. The sulfite levels typically used to protect wine are toxic to typical malolactic organisms. While undergoing malolactic fermentation, the wine may be vulnerable to oxidation and/or microbial spoilage. Hence, winemakers typically monitor malolactic fermentation carefully and apply sulfite soon after the malic acid levels are reduced to the target level. Adding sulfite too soon would inhibit the malolactic fermentation and leave undesirable amounts of malic acid in the wine. Adding sulfite too late would unnecessarily expose the wine to spoilage. Determining levels of malic acid has been a complex, qualitative, and/or expensive process. Thus, there has been a long felt need for an inexpensive, accurate method of determining malic acid levels in solutions such as wine and grape musts.

The determination of malate levels in food, beverages, and the winemaking process is important to product quality control and smooth completion of fermentation. Typically, such analysis is accomplished by chromatography, or by enzymatic/spectrophotometric assay. One commonly used method is paper chromatography, in which a suitable filter paper is spotted with microliter amounts of wine samples and with reference solutions of the organic acids to be separated: typically, lactic, malic and tartaric acid. After drying, the paper is placed in a developing tank where a solution of butanol, water and formic acid is drawn up the paper by wicking action. As the solvent front proceeds up the paper, the various organic acids migrate various characteristic distances up the paper as well. At the end of this stage, which takes 6 to 24 hours, the separated acids can be visualized with bromocresol green as yellow spots on a light green background. In this method, malic acid can be determined semi-quantitatively at best. Another method that can be used is high performance ion exchange liquid chromatography (HPLC), in which organic acids elute in characteristic order from an ion exchange column and are detected and quantified by a suitable photometric detector.

The most common approach to detection of malic acid is spectrophotometric assay using enzymatic production of NADH (nicotinamide adenine dinucleotide hydride) that occurs when malate dehydrogenase (MDH) acts on I-malate and NAD (nicotinamide adenine dinucleotide). The NADH can be measured by the increase in absorbance at 340 nm. Typically, colored compounds interfere with spectrophotometric assays and hence are generally removed (e.g., red wine is clarified before assaying). Since the equilibrium of the MDH reaction shown below lies to the left, a second enzyme, glutamate oxaloacetate transaminase (GOT) is added to facilitate the removal of oxaloacetate as it is formed.

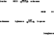

However, there are several difficulties in the use of the above-mentioned methods. As already mentioned, the paper chromatography method, though requiring only relatively simple equipment, is a lengthy process that is tedious and essentially qualitative, making it difficult to assess whether malic acid levels have completely achieved the predetermined level (typically the target is less than 0.2 mg/mL). In contrast, the HPLC method is quantitative and relatively quick, but the cost and required expertise for operating the method are very high. Similarly, the spectrophotometric/enzymatic assay is quantitative and of intermediate duration, but again the expertise and equipment requirements are high.

Another method that has been used in the past is detection of malic acid by microbial assay. An example of this method is given by the article “Manometric Determination of I(-) Malic Acid in Grape Musts and Wines” by G. F. Kolar, Am. J. Enol. Vitic. 13:99-104 (1962). In this method, malic acid is converted into CO₂ and lactic acid by Lactobacillus plantarum, and the evolved CO₂ is measured manometrically using a glass system known as a Warburg apparatus. The reaction is:

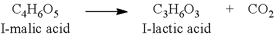

It is now known that this decarboxylating transformation is carried out by a single protein, malolactic enzyme, which can be purified from L. plantarum in fully active form as described in “Malolactic enzyme of Lactobacillus plantarum. Purification, properties, and distribution among bacteria” by G. Caspritz and F. Radler, J. Biol. Chem. 258:4907-4910 (1983). Similar enzymes are now known to exist in several lactobacillus species and in the malolactic bacterium O. oeni that is used in the winemaking process; generally these require cofactors NAD and manganese.

There are other enzymes and biological systems known to carry out decarboxylation of I-malic acid. For example, malic enzyme (e.g., Enzyme Commission numbers EC 1.1.1.38, EC 1.1.1.39, and EC 1.1.1.40, also known as malate dehydrogenase (oxaloacetate-decarboxylating), malate dehydrogenase (decarboxylating), malate dehydrogenase (oxaloacetate-decarboxylating) (NADP⁺)) carries out the conversion of malic acid to pyruvate and CO₂, requiring NAD⁺ as co-substrate:

(S)-malate+NAD⁺ [symbol] pyruvate+CO₂+NADH

See for example, “Studies on regulatory functions of malic enzymes. V. Comparative studies of malic enzymes in bacteria.” by Iwakura M., Tokushige M., Katsuki H., Muramatsu S., J. Biochem. 83(5):1387-94 (1978).

The manometric method for analysis of malic acid in wines was used frequently until supplanted by chromatographic and spectrophotometric methods (mentioned above) during the 1970s and later. There are several reasons why the manometric method is difficult to use routinely. One is the difficulty of obtaining reliable manometric measurements, a time-consuming process. The Warburg apparatus is a rather complicated assembly of glass vessels, tubing, valves and ground glass fittings. One of these fittings allows the device to be attached to a valve that leads to a mercury manometer, while one or more other fittings allows the introduction of reactants that can be added at different stages in the assay process. The Warburg apparatus is fragile and costly, and its use requires considerable training and expertise. Another difficulty in the manometric method was the difficulty in maintaining viable cultures of L. plantarum, which must be kept at the right level of temperature and growth phase to give reliable results.

### SUMMARY

Analytical methods and devices according to the present disclosure may be used to assay the concentration of malic acid, or related species, in a sample, e.g., preparations of fruit, vegetables, juice, and/or wine. Methods comprise combining a liquid sample with a reaction mixture, incubating the combined liquid sample and reaction mixture, and measuring a parameter characteristic of the resulting incubated mixture. During the incubation, the reaction mixture generally reacts with the liquid sample to transform malic acid or related species that is present in the liquid sample, and in the process generates and/or consumes CO₂, hydrogen ions, and/or hydroxide ions. The measured parameter may be pressure, pH, an amount (e.g., volume, mass) of CO₂ generated during the incubation, a parameter related to the foregoing, and/or any other parameter related to the amount of malic acid or related species transformed.

Systems comprise a device, a kit, and/or reagents configured to assay malic acid, or related species, in a liquid sample. Systems may be used to assay malic acid or related species by exploiting the fact that malic acid, or related species, may be transformed by enzymes, the transformation process generating and/or consuming CO₂, hydrogen ions, and/or hydroxide ions.

### BRIEF DESCRIPTION OF THE DRAWINGS

FIG. 1 is a schematic representation of methods according to the present disclosure.

FIG. 2 is a schematic representation of systems according to the present disclosure.

FIG. 3 is a response of pressure to malic acid using analytical methods and devices according to the present disclosure.

FIG. 4 is a response of pH to malic acid using analytical methods and devices according to the present disclosure.

### DESCRIPTION

Analytical methods and devices according to the present disclosure may be used to assay the concentration of malic acid, or related species (e.g., malate), within a sample, e.g., preparations of fruit, vegetables, juice, and/or wine. Liquid samples are combined with a reaction mixture, incubated, and a parameter characteristic of the resulting incubated mixture is measured. During the incubation, the reaction mixture generally reacts with the liquid sample to transform malic acid or related species that is present in the liquid sample, and in the process generates and/or consumes CO₂, hydrogen ions, and/or hydroxide ions. If the liquid sample has essentially no malic acid or related species, the reaction mixture may not significantly react with the liquid sample. The measured parameter may be pressure, pH, an amount (e.g., volume, mass) of CO₂ generated during the incubation, a parameter related to the foregoing, and/or any other parameter related to the amount of malic acid or related species transformed.

Though some malic acid transformation reactions are known to generate CO₂, and thus manometric assays have been used previously, pH change due to the reaction was unexpected. For example, the classical stoichiometry of the malolactic transformation shows no generation or consumption of acid or base (C₄H₆O₆→C₃H₆O₃+CO₂). But, by considering the reaction as it occurs where malic and lactic acids are primarily in their deprotonated forms, one gets the surprising result that pH does increase because hydroxide ion is released:

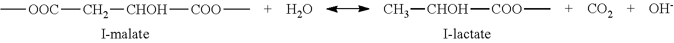

An acid is primarily in its deprotonated form at a pH greater than about 1 pH unit higher than the acid's pKa. At plus one unit, an acid is approximately 90% deprotonated. At plus two units, an acid is approximately 99% deprotonated. Malic acid has a pKa of about 3.4 and lactic acid has a pKa of about 3.9. Thus, both acids are primarily in their deprotonated forms at a pH of greater than about 4.9, greater than about 5.5, greater than about 6, or greater than about pH 6.3.

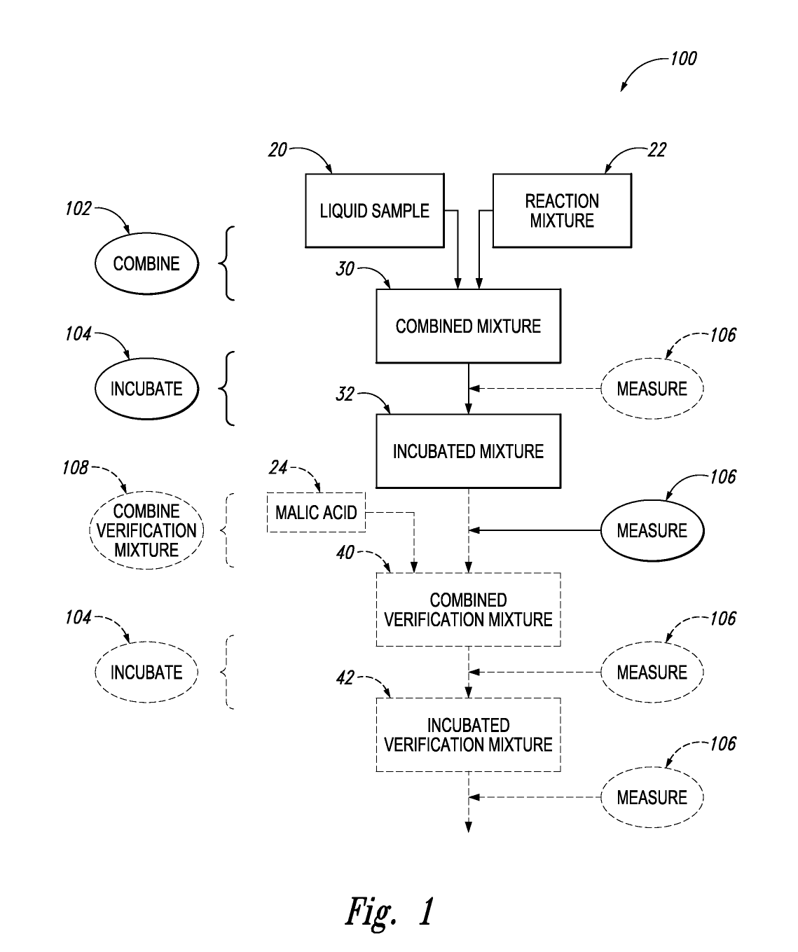

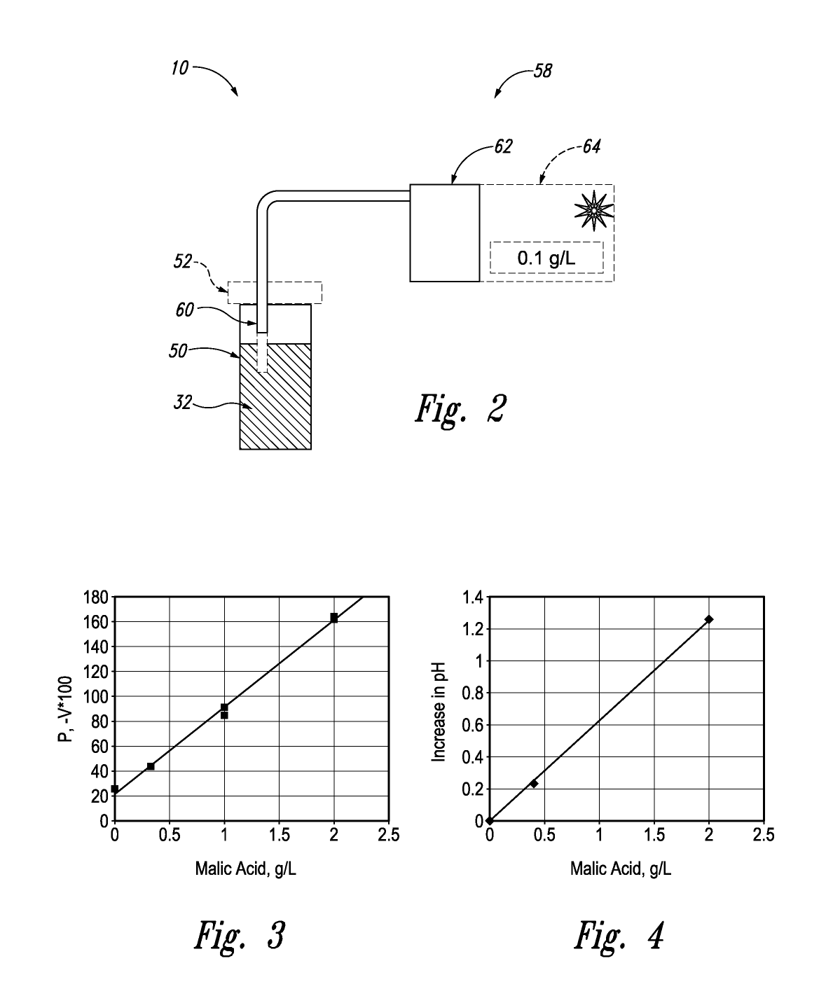

FIGS. 1-2 illustrate analytical methods and devices for malic acid analysis and elements thereof according to the present disclosure. Elements that serve a similar, or at least substantially similar, purpose are labeled with numbers consistent among the figures. Like numbers in each of the figures, and the corresponding elements, may not be discussed in detail herein with reference to each of the figures. Similarly, all elements may not be labeled in each of the figures, but reference numerals associated therewith may be used for consistency. Elements, components, and/or features that are discussed with reference to one of the figures may be included in and/or used with any of the elements, components, and/or features of another figure without departing from the scope of the present disclosure. In general, elements that are likely to be included are illustrated in solid lines, while elements that may be optional or alternatives are illustrated in dashed lines. However, elements that are shown in solid lines are not necessarily essential, and an element shown in solid lines may be omitted without departing from the scope of the present disclosure.

As schematically illustrated in FIG. 1, methods 100 comprise combining 102 a liquid sample 20 and a reaction mixture 22 to create a combined mixture 30. After combining 102, methods 100 comprise incubating 104 the combined mixture to create an incubated mixture 32. Methods 100 further comprise measuring 106 a measured parameter characteristic of the incubated mixture and/or the combined mixture. The reaction mixture is configured to generally react with the liquid sample if malic acid or a related species is present. When the liquid sample and the reaction mixture are combined, malic acid, or related species, may be transformed, producing and/or consuming other species, e.g., CO₂, hydrogen ions, and/or hydroxide ions. The parameter to be measured may be selected to indicate the presence and/or extent of any reaction between the reaction mixture and the liquid sample.

Combining 102 generally includes contacting the liquid sample 20 with the reaction mixture 22 in a manner to allow the components to react. The liquid sample may be added to the reaction mixture or vice versa. Components of the reaction mixture may be added to the liquid sample and/or vice versa.

The liquid sample 20 may be any substance in which malic acid or related species may be present. The liquid sample may be a liquid, a mixture, a solution, a suspension, a slurry, and/or a paste. For example, the liquid sample may be fruit juice, vegetable juice, wine, and/or grape must.

Typically, combining 102 includes combining, in a container 50 (as illustrated in FIG. 2), a total volume of less than 20 mL, 10 mL, 8 mL, 6 mL, 5 mL, 4 mL, 3 mL, 2 mL, or 1 mL, and/or greater than 0.1 mL, 0.2 mL, 0.5 mL, or 1 mL. The container may have a maximum volume equal to or greater than the total volume combined. The liquid sample 20 may have a volume of less than 10 mL, 8 mL, 6 mL, 5 mL, 4 mL, 3 mL, 2 mL, or 1 mL. As an example of suitable volumes, the liquid sample 20 and the reaction mixture 22 combined may be about 2-3 mL and placed into a 4 mL container. A measured amount of liquid sample and/or reaction mixture (or its components) may be placed in and/or added to the container.

The reaction mixture 22 includes a source of enzyme to chemically transform malic acid, or related species, in the liquid sample. The reaction mixture generally includes a liquid and may include components that are liquids, solids, particulates, capsules, vesicles, and/or cells. A source of enzyme to transform malic acid is a composition that includes an appropriate enzyme, e.g. a malolactic enzyme and/or a malic enzyme. An enzyme as used herein, unless otherwise noted, is an enzyme to catalyze a chemical reaction in which malic acid and/or a related species participates, e.g., malic acid is converted to a new species and/or is consumed in the reaction. Example transformations include dehydrogenation and/or decarboxylation. Hence, enzymes may be described as dehydrogenases and/or decarboxylases.

The source of enzyme may include a suspension and/or a dry powder of live, inactivated, and/or dead (e.g., permeabilized, irradiated, freeze-dried) bacteria (e.g., lactobacteria), yeast, and/or other organisms, or cells thereof (including tissues), that produce and/or contain the enzyme. Illustrative, non-exclusive example organisms include L. plantarum, O. oeni, Saccharomyces cerevisiae, and Schizosaccharomyces pombe. Suitable enzyme also may be produced by a genetically-modified organism (e.g., recombinant malolactic enzyme). Where live organisms are used, the reaction mixture 22 may include carbon sources and/or nutrients (e.g., glucose).

The source of enzyme may include an enzyme purified from L. plantarum, O. oeni, and/or other organisms that produce and/or contain the enzyme, and/or a recombinant version thereof. For example, recombinant forms of malolactic are described in “Malolactic enzyme from Oenococcus oeni: Heterologous expression in Escherichia coli and biochemical characterization” by C. Schümann, H. Michlmayr, A. M. del Hierro, K. D. Kulbe, V. Jiranek, R. Eder, and T.-H. Nguyen, Bioengineered 4(3):147-152 (2013).

Enzymes to transform malic acid and/or related species typically require cofactors (including cosubstrates) such as NAD⁺, or equivalent, and/or manganese. Living organisms that produce or otherwise contain such an enzyme may include some or all of the necessary cofactors. Where cofactors are required and/or useful, the reaction mixture 22 may include cofactors. For example, the source may be a suspension of L. plantarum and/or O. oeni that has been permeabilized, e.g., by treatment with lysozyme, as in “Biochemical Basis for Glucose-Induced Inhibition of Malolactic Fermentation in Leuconostoc oenos” by M. Miranda, A. Ramos, M. Veiga-da-Cunha, M. C. Loureiro-dias, and H. Santos, J. Bacteriol. 179(17):5347-5354 (1997). As another example, the source of enzyme may include purified enzyme and cofactors.

Enzyme activity may be described in enzyme units, U, where 1 U is the amount of enzyme required to produce product (and/or consume reactant) at a rate of 1 μmol per minute at 25° C. For enzyme as described herein, the reactant may be malic acid and/or related species and the product may be lactic acid, CO₂, and/or related species. The reaction mixture 22 and/or the combined mixture 30 may have an enzyme activity of at least about 0.5 U, at least about 1 U, or at least about 2 U, less than about 10 U, less than about 5 U, or less than about 3 U, and/or about 1 U or about 2 U. Additionally or alternatively, the reaction mixture and/or the combined mixture may have an enzyme activity concentration of greater than 0.1 U/mL, 0.2 U/mL, 0.3 U/mL, 0.4 U/mL, 0.5 U/mL, 0.6 U/mL, 0.7 U/mL, 0.8 U/mL, or 0.9 U/mL.

The reaction mixture 22 and/or the combined mixture 30 also may include solvent, buffering agent, buffer, acid, base, and/or cofactors (e.g., NAD+, manganese, and/or related species). For example, the reaction mixture may include about 100 nmol of NAD per unit of enzyme activity. Illustrative, non-exclusive example buffers include citrate buffer and acetate buffer.

Methods 100 may comprise adjusting the pH of the combined mixture 30 to achieve suitable conditions for enzyme activity. For example, the reaction mixture 22 may include acid, base, and/or buffer sufficient to adjust the pH of the combined mixture 30 to greater than about pH 5, about pH 5.5, about pH 6, about pH 6.3, or about pH 6.5.

After creating the combined mixture 30, methods 100 comprise incubating 104 the combined mixture to allow the enzymatic transformation of malic acid or related species. Generally, incubating includes allowing the reaction to proceed at a suitable temperature (e.g., 10-45° C., 10-35° C., 15-25° C., about 20° C., or about 25° C.) and for a suitable time (e.g., greater than 5 min., 10 min., 15 min., 20 min., 30 min., 40 min., 50 min., or 60 min. and/or less than 90 min., 60 min., 50 min., 40 min., 30 min., or 20 min.). Suitable time and temperature may be estimated by the enzymatic activity of the combined mixture. During the incubating 104, the combined mixture may be mixed and/or stirred a single time, periodically, or continuously.

Methods 100 comprise measuring 106 a measured parameter characteristic of the incubated mixture and/or the combined mixture. The measured parameter may be the amount (e.g., volume, pressure, and/or mass) of CO₂ generated, the amount of gas generated, the pH generated, and/or a parameter related to the foregoing. The measuring 106 generally is performed after the incubating 104 and may be performed before and/or at least partially concurrently with the incubating 104. The measured parameter may be a difference in quantity between the incubated mixture and the combined mixture. For example, the measured quantity may be the pH change from the combined mixture to the incubated mixture. Further, methods 100 may comprise ceasing the incubating when the measured parameter and/or a rate of change of the measured parameter is below, above, or approximately at a predetermined threshold.

Generally, measuring 106 may be performed, directly or indirectly, with conventional methods, typically including contacting the incubated mixture 32, combined mixture 30, and/or gas evolved from at least one of the mixtures. For example, the pH of the incubated mixture may be monitored and/or measured with a pH probe, a pH meter, a pH strip, etc. in contact with the incubated mixture and/or the combined mixture. As another example, the pH may be measured by titrimetry with standard acid or standard base (e.g., adding a measured amount of acid or base to achieve a standard pH value).

As a further measuring 106 example, the amount (e.g., pressure, volume, mass) of gas emitted during the incubation may be measured with a gas probe in contact with a container holding the combined mixture 30 and/or incubated mixture 32. Methods 100 which include measuring 106 gas properties may include sealing (e.g., hermetically sealing) a container that holds the combined mixture and/or the incubated mixture. Measuring may include placing a gas probe in contact with the gas in the container (e.g., by piercing the container).

Methods 100 may comprise correlating the measured parameter and the malic acid concentration of the liquid sample 20 (e.g., by measuring, establishing, and/or calculating a relationship between malic acid concentration and the measured parameter). Methods 100 may comprise calculating the malic acid concentration of the liquid sample based upon the measured parameter (e.g., by a measured, established, and/or calculated relationship between malic acid concentration and the measured parameter). Methods 100 may comprise determining whether the malic acid concentration in the liquid sample is below, above, or approximately at a predetermined threshold based upon the measured parameter.

Methods 100 may comprise one or more steps to remove gas and/or CO₂ from the liquid sample 20 and/or at least a component of the combined mixture 30. For example, methods 100 may comprise degassing the liquid sample prior to the combining 102, e.g., by shaking and/or boiling the liquid sample. Boiling may include bringing the liquid sample to a rolling boil for at least 5 sec., 10 sec., 15 sec., 20 sec., 30 sec., 40 sec., 50 sec., or 60 sec. If the liquid sample is boiled before combining 102 and/or incubating 104, the liquid sample may be cooled before the combining 102 and/or incubating 104, and may be cooled to less than 60° C., 45° C., 40° C., 35° C., 30° C., 25° C., or 20° C.

Methods 100 may comprise removing, inactivating, reducing, and/or suppressing compounds in the liquid sample 20 and/or the combined mixture 30 that may interfere with the enzyme. Removing, inactivating, reducing, and/or suppressing typically is performed before incubating 104, but may be performed at least partially concurrently with incubating 104. Interfering compounds may include tannins, as are present in red wine, and high concentrations of alcohol (e.g., above about 14%). Removing, inactivating, reducing, and/or suppressing may include incubating the liquid sample and/or the combined mixture with a solid phase composition including one or more of ion exchange compositions (e.g., reversed-phase sorbent material such as C18 and/or C8 silica), reversed-phase compositions, and PVPP (polyvinylpyrrolidone, a polymeric material used for clarification of wines) compositions. The removing, inactivating, reducing, and/or suppressing may further include separating the solid phase composition from the liquid sample and/or the combined mixture before incubating 104. For example, interfering compounds may be separated by passing 5 mL of wine through a 1 cm column packed with a reversed-phase sorbent material. The liquid which elutes immediately contains malic acid and related species, while interfering compounds are retained on the column. As another example, interfering compounds may be separated by mixing about 0.2 g of solid PVPP particles with about 3 mL of wine, allowing the materials to mix and/or stir for about 15 minutes. Interfering compounds will bind to the PVPP particles, while malic acid and related species stay in solution. Further separation may be achieved by allowing the PVPP particles to settle and using the supernatant as the liquid sample for the methods 100.

Methods 100 may comprise verifying (e.g., calibrating and/or validating) the prior combining 102, incubating 104, and/or measuring 106. Verifying may include adding malic acid or related species (together indicated by 24) to the incubated mixture 32 after the measuring 106. Methods 100 may not exhaust the enzyme and/or cofactors during the incubating 104 and, therefore, further enzymatic reaction may be performed with the enzyme and/or cofactors remaining after the incubating 104. The added malic acid or related species may react with the remaining enzyme and/or cofactors after the incubating 104 to verify that the reaction mixture (including the enzyme) is active and/or that the prior combining 102 and incubating 104 were performed correctly.

Generally, verifying may include combining 108 a verification mixture by adding a measured amount of malic acid or related species to the incubated mixture 32, typically after measuring 106, to create a combined verification mixture 40. Verifying may include incubating 104 the combined verification mixture to create an incubated verification mixture 42. Verifying may further include measuring 106 a measured verification parameter characteristic of the incubated verification mixture. The measured verification parameter may be, or may be related to, at least one of pressure, pH, and an amount of CO₂ generated during the incubating 104 of the combined verification mixture. Generally, the measured parameter characteristic of the incubated mixture and the measured verification parameter characteristic of the incubated verification mixture are the same type of parameter (e.g., both are pH).

Methods 100 may comprise indicating the amount (e.g., concentration, mass) of the malic acid or related species that was originally present in the liquid sample. Indicating may include indicating whether the measured parameter was above, below, and/or approximately at a predetermined threshold. Indication may be by visual indicator (e.g., numeric indicator, light, etc.), audible indicator, and/or tactile indicator.

FIG. 2 illustrates systems 10 for analysis of malic acid or related species. Systems 10 comprise a device, a kit, and/or reagents configured to assay malic acid, or related species, in a liquid sample. Systems 10 may be used to assay malic acid or related species by exploiting the fact that malic acid, or related species, may be transformed by enzymes, the transformation process generating and/or consuming CO₂, hydrogen ions, and/or hydroxide ions.

Systems 10 may comprise a device 58 that includes a probe 60, a transducer 62, and/or an indicator 64. The device 58 may be configured to measure and indicate a parameter characteristic of a liquid (e.g., the incubated mixture 32) contained in a container 50 and/or a reaction in the liquid. The parameter may be the amount (e.g., volume, pressure, and/or mass) of CO₂ present and/or generated, the amount of gas present and/or generated, the pH, the generated pH, and/or a parameter related to the foregoing. Where the device is configured to measure pH, the device typically is configured to measure pH in the range of at least 4-7, with an accuracy and/or a precision of better than 0.1, 0.05, or 0.02 pH units. Where the device is configured to measure pressure, the device typically is configured to measure differential pressure in the range of about 0-50 kPa, or 0-10 kPa, with an accuracy and/or a precision of better than 1 kPa, 0.5 kPa, 0.2 kPa, or 0.1 kPa. Illustrative, non-exclusive examples of devices include a pH meter, a pressure gauge, a Vinmetrica SC-100 Sulfite Wine Analyzer, a Vinmetrica SC-100A Sulfite Wine Analyzer, a Vinmetrica SC-300 Sulfite and pH/TA Wine Analyzer, and a Vinmetrica SC-50 MLF Analyzer.

The probe 60 is configured to measure a parameter characteristic of a liquid contained in a container 50 and/or a reaction in the liquid. The parameter may be the amount of CO₂ present and/or generated, the amount of gas present and/or generated, the pH, the generated pH, and/or a parameter related to the foregoing. The probe may be configured to contact the container, the liquid, and/or gas within the container. The probe may include a needle configured to pierce the container.

The transducer 62 is configured to transform a signal from the probe (e.g., an electrical signal, a gas sample) into an electrical signal processed by the device. Transducers may include amplifiers, voltage-to-current transducers, sensors, flow sensors, and/or pressure transducers. Illustrative, non-exclusive examples of pressure transducers include pressure sensors, differential pressure transducers, Motorola MPX2050DP, and Motorola MPX2010DP. As another example, a pressure sensor configured to detect CO₂ produced in an enzymatic assay is described in “Refinement of the Pressure Assay for Milk Urea Nitrogen” by D. M. Jenkins, M. J. Delwiche, E. J. DePeters, and R. H. BonDurant, J. Dairy Sci. 83:2042-2048 (2000).

The indicator 64 may include a visual indicator (e.g., numeric indicator, light, etc.), audible indicator, and/or tactile indicator. The indicator may be configured to indicate the electrical signal produced by the transducer, a signal and/or value related to the parameter measured by the probe and/or transducer, and/or a signal and/or value related to the amount (e.g., concentration, mass) of the malic acid or related species that was, or is, present in the liquid. The indicator may be configured to indicate whether the electrical signal, the parameter, and/or the amount was, or is, above, below, and/or approximately at a predetermined threshold.

The container 50 is configured to hold and/or contain the liquid while the liquid is undergoing one or more of malic acid or related species the reactions described herein. The container may include a cap 52 with an optional septum. The cap and/or septum may include an elastomer, a rubber, and/or a plastic. The cap may be configured to seal (e.g., hermetically seal) the container.

### EXAMPLE 1

### Pressure-Based Assay

FIG. 3 is a graph of malic acid standards for a pressure-based assay. Procedure for this illustrative, non-exclusive example pressure-based assay:
1. Preparation of samples: Samples are boiled briefly to expel any CO₂ present. 10-25 mL wine is placed in a small vessel and heated in a microwave oven on high for 45 sec., then allowed to cool. The pH of the sample is adjusted to 5.0 with 2M NaOH. Wine samples are diluted 1 to 1 with water at this point.
2. Standard concentrations of I-malic acid, ranging typically from 0.1 to 5 g/L, can be prepared in water, adjusted to pH 5.0 also.
3. 1.6 mL of sample or standard is placed in a 4 mL vial (13-425 screw-cap type).
4. 0.2 mL of a water solution that includes 100 mM d-glucose, 5 mg/mL MnCl₂, and 5 mg/mL KH₂PO₄, is added to each vial.
5. 0.050 mL of 3M acetate/acetic acid, pH 5.0, is added to each vial.
6. 0.10 mL of a buffered suspension of L. plantarum, containing about 8 mg dry weight of freshly harvested bacteria, is added to each vial and the vial immediately tightly capped with a screw-cap fitted with 6 mm silicone septum. Alternatively, 0.10 mL of a buffered solution containing about 1 U of malolactic enzyme and about 100 nmol of NADP can be added.
7. Vials are shaken gently every 5 minutes for 30 minutes.
8. Each vial is shaken vigorously for 10 seconds, then pressure is read by piercing the septum with the device's measuring needle.
9. If validation/calibration is to be done, the sample is first re-read after 15 minutes to ensure that no further increase in pressure has occurred and that therefore the previous reaction is finished. Then the vial is opened and 0.02 mL of 100 mg/mL I-malic acid is added. The vial is recapped and steps 7 and 8 above are repeated.

The foregoing procedure can also be run using vials preloaded with a source of malolactic enzyme. This enzyme can be added in advance as a solution of enzyme or a bacterial suspension in a volume of about 0.1 mL, or it can be present as a lyophilized powder of enzyme, bacterial suspension, or permeabilized bacteria, that is reconstituted with about 0.1 mL water prior to the assay, or with the sample itself at the beginning of the assay. In either case, step 6 above is omitted and step 3 is performed after step 5.

### EXAMPLE 2

### pH-Based Assay

FIG. 4 is a graph of malic acid standards for a pH-based assay. Procedure for this illustrative, non-exclusive example pH-based assay:
1. Preparation of samples: 15 mL wine is placed in a small vessel. The pH of the sample is adjusted to about 5.5 by adding 1 mL of 1M NaOH.
2. The samples are heated in a microwave oven on medium power for 60 sec., to bring them to a boil, then allowed to cool.
3. 1 mL of 0.225 M acetate buffer, pH 5.5, containing 2.5 mg MnCl₂.4H₂O, is added to the cooled samples, then the volume is made up to 20 mL with water. An initial pH measurement is made and recorded.
4. If desired, standard concentrations of I-malic acid, ranging typically from 0.1 to 5 g/L, can be prepared in water, adjusted to pH 5.5 also and carried through steps 1-3 above.
5. 2.0 mL of sample or standard is placed in a 4 mL vial (13-425 screw-cap type).
6. 0.10 mL of a buffered suspension of L. plantarum, containing about 10 mg dry weight of freshly harvested bacteria, is added to each vial and the vial is capped. Alternatively, 0.10 mL of a buffered solution containing about 2 U of malolactic enzyme and about 100 nmol of NAD can be added.
7. The vials are incubated for a period of time of about 30-60 min., with occasional shaking.
8. At the end of the incubation time, the vials are shaken again, then the pH is measured with a small pH probe inserted directly into the vial.

The foregoing procedure can also be run using vials preloaded with a source of malolactic enzyme. This enzyme can be added in advance as a solution of enzyme or a bacterial suspension in a volume of about 0.1 mL, or it can be present as a lyophilized powder of enzyme, bacterial suspension, or permeabilized bacteria, that is reconstituted with about 0.1 mL water prior to the assay, or with the sample itself at the beginning of the assay. In either case, step 6 above is omitted and step 3 is run after step 5.

### EXAMPLE 3

### Verification

The analytical methods and devices of the present disclosure may be configured, and/or include a step, to check validity of an assay. After incubating 104 and measuring 106, samples that appear to have had little to no change in measured parameter may be checked for validity by adding malic acid, or related species, to these putatively negative samples. If they are truly negative samples, the additional malic acid, or related species, should cause an appropriate parameter value increase to occur after a second incubation step. Table 1 gives an example of the results of a verification step with a pH-based assay.

Table I

| | Time | | |
|---|---|---|---|
| | | | 60 min. |
| | | | after spike |
| Wine Sample | 0 min. | 60 min. | (120 min.) |
| Red wine #1 | 5.25 | 5.29 | 5.68 |
| Red wine #2 | 5.39 | 5.42 | 5.86 |
| Red wine #3 | 5.23 | 5.22 | 5.69 |

At time T=0 minutes, before addition of any enzyme source, the initial pH of each wine is recorded. Then the enzyme source (in the form of O. oeni bacteria) is added. After 60 minutes of incubation, the pH of the three incubated wine mixtures is measured. At this point, the pH has not changed significantly because there is little malic acid present (all samples were <0.3 g/L as analyzed separately by spectrophotometric enzyme assay). To verify the putative negative results, 1M malic acid (134 g/L), adjusted to pH 5.5, is added to the samples to make the malic acid concentration 2 g/L, and the reaction allowed to proceed another 60 minutes. The pH increases during the second incubation as expected, showing that the first incubation produced valid negative results. No pH changes were seen when the same samples and spiked samples were analyzed without O. oeni added to them.

### EXAMPLES OF ANALYTICAL METHODS AND DEVICES

Illustrative, non-exclusive examples of analytical methods and devices for malic acid analysis according to the present disclosure are described in the following enumerated paragraphs:

A1. A method of detecting malic acid, or related species, in a liquid sample comprising:

combining a liquid sample and a reaction mixture to create a combined mixture;

incubating the combined mixture to create an incubated mixture; and

measuring a measured parameter characteristic of the incubated mixture;

wherein the reaction mixture includes a source of enzyme to transform malic acid or related species.

A2. The method of paragraph A1, wherein the liquid sample is less than 10 mL, 8 mL, 6 mL, 5 mL, 4 mL, 3 mL, 2 mL, or 1 mL.

A3. The method of any of paragraphs A1-A2, wherein an activity of the enzyme in the combined mixture is at least about 0.5 U, at least about 1 U, or at least about 2 U, less than about 10 U, less than about 5 U, or less than about 3 U, and/or about 1 U or about 2 U.

A4. The method of any of paragraphs A1-A3, wherein the activity concentration of the enzyme in the combined mixture is greater than 0.1 U/mL, 0.2 U/mL, 0.3 U/mL, 0.4 U/mL, 0.5 U/mL, 0.6 U/mL, 0.7 U/mL, 0.8 U/mL, or 0.9 U/mL.

A5. The method of any of paragraphs A1-A4, wherein the combining includes placing a measured amount of the liquid sample into a container.

A6. The method of any of paragraphs A1-A5, wherein the reaction mixture includes buffer.

A7. The method of any of paragraphs A1-A6, wherein the reaction mixture includes at least one of manganese and NAD⁺.

A8. The method of any of paragraphs A1-A7, wherein the enzyme is a malolactic enzyme from any of organism that contains malolactic enzyme, optionally wherein the organism is selected from the group consisting of L. plantarum, O. oeni, S. cerevisiae, and S. pombe.

A9. The method of any of paragraphs A1-A8, wherein the enzyme is a malic enzyme from any of organism that contains malic enzyme.

A10. The method of any of paragraphs A1-A9, wherein the source of enzyme is a suspension of microbes.

A11. The method of any of paragraphs A1-A10, wherein the source of enzyme is a preparation of permeabilized microbes.

A12. The method of any of paragraphs A1-A11, wherein the source of enzyme is a cell-free extract.

A13. The method of any of paragraphs A1-A12, wherein the combining includes placing the liquid sample and/or combined mixture in a container, and further comprising sealing the container after the combining and before the incubating.

A13.1. The method of paragraph A13, wherein the sealing includes hermetically sealing the container with a septum that includes at least one of an elastomer, a rubber, and a plastic, and optionally wherein the measuring includes piercing the septum.

A14. The method of any of paragraphs A1-A13.1, further comprising adjusting, before the incubating, a pH of the combined mixture to greater than about pH 5, about pH 5.5, about pH 6, about pH 6.3, or about pH 6.5, and optionally wherein the adjusting includes adding at least one of a buffer and a base.

A15. The method of any of paragraphs A1-A14, wherein the incubating includes incubating for greater than 5 min., 10 min., 15 min., 20 min., 30 min., 40 min., 50 min., or 60 min. and/or less than 90 min., 60 min., 50 min., 40 min., 30 min., or 20 min.

A16. The method of any of paragraphs A1-A15, wherein the incubating includes mixing the combined mixture during the incubating.

A17. The method of any of paragraphs A1-A16, wherein the measuring includes measuring a parameter related to an amount of CO₂ generated during the incubating and optionally wherein the measured parameter is the amount of CO₂ generated.

A18. The method of any of paragraphs A1-A17, wherein the measuring includes measuring a pressure and/or a parameter related to pressure of a/the container holding the incubated mixture, and optionally wherein the measured parameter is the pressure and/or a parameter related to pressure.

A19. The method of any of paragraphs A1-A18, wherein the measuring includes measuring a pH and/or a parameter related to pH of the incubated mixture, and optionally wherein the measured parameter is the pH and/or a parameter related to pH.

A20. The method of any of paragraphs A1-A19, wherein the measuring includes measuring a pH of the combined mixture before the incubating and after the incubating, and wherein the measuring includes calculating a differential pH change associated with the incubating, and optionally wherein the measured parameter is the differential pH change.

A21. The method of any of paragraphs A1-A20, wherein the measuring includes measuring the measured parameter at least partially concurrently with the incubating.

A21.1. The method of paragraph A21, further comprising ceasing the incubating when the measured parameter and/or a rate of change of the measured parameter is below, above, or approximately at a predetermined threshold.

A22. The method of any of paragraphs A1-A21.1, further comprising correlating the measured parameter and the malic acid or related species concentration of the liquid sample.

A23. The method of any of paragraphs A1-A22, further comprising calculating a malic acid or related species concentration of the liquid sample based upon the measured parameter.

A24. The method of any of paragraphs A1-A23, further comprising determining whether a malic acid or related species concentration in the liquid sample is below, above, or approximately at a predetermined threshold based upon the measured parameter.

A25. The method of any of paragraphs A1-A24, further comprising boiling the liquid sample prior to the combining.

A25.1. The method of paragraph A25, further comprising cooling the liquid sample to less than 60° C., 45° C., 40° C., 35° C., 30° C., 25° C., or 20° C. after the boiling and prior to the combining.

A25.2. The method of any of paragraphs A25-A25.1, wherein the boiling includes bringing the liquid sample to a rolling boil for at least 5 sec., 10 sec., 15 sec., 20 sec., 30 sec., 40 sec., 50 sec., or 60 sec.

A26. The method of any of paragraphs A1-A25.2, further comprising degassing the liquid sample prior to the combining, optionally wherein the degassing includes shaking and/or boiling the liquid sample.

A27. The method of any of paragraphs A1-A26, further comprising removing compounds from the liquid sample and/or the combined mixture that interfere with the enzyme to transform malic acid or related species, and optionally wherein the removing is performed before the incubating.

A27.1. The method of paragraph A27, wherein the removing includes incubating the liquid sample and/or the combined mixture with a solid phase composition including one or more of ion exchange compositions, reversed-phase compositions, and PVPP compositions, optionally wherein the removing includes separating the solid phase composition from the liquid sample and/or the combined mixture before the incubating the combined mixture.

A27.2. The method of any of paragraphs A27-A27.1, wherein the removing includes reducing a concentration of the compounds.

A27.3. The method of any of paragraphs A27-A27.2,wherein the removing includes reducing a concentration of tannins in the liquid sample and/or the combined mixture.

A28. The method of any of paragraphs A1-A27.2, further comprising validating the measured parameter by

adding a measured amount of malic acid or related species to the incubated mixture to create a combined verification mixture;

incubating the combined verification mixture to create an incubated verification mixture; and

measuring a measured verification parameter characteristic of the incubated verification mixture;

wherein the measured verification parameter is, and/or is related to, at least one of pressure, pH, and an amount of CO₂ generated during the incubating the combined verification mixture.

B1. A kit for detecting malic acid in a sample solution, the kit comprising:

a first container that includes a source of enzyme to transform malic acid;

a second container that includes a buffer solution, and manganese.

B2. The kit of paragraph B1, wherein the source of enzyme includes one or more enzymes selected from the group consisting of a malic enzyme, a malolactic enzyme, a malate dehydrogenase, and a malic acid decarboxylase.

B3. The kit of any of paragraphs B1-B2, wherein at least one of the first container and the second container include NAD⁺.

B4. The kit of any of paragraphs B1-B3, wherein the first container is a reaction vessel configured to hold a liquid sample.

B4.1. The kit of paragraph B4, wherein the reaction vessel is configured to seal the liquid sample within the reaction vessel.

B4.2. The kit of any of paragraphs B4-B4.1, wherein the reaction vessel includes a piercable septum.

B5. The kit of any of paragraphs B1-B4.2, wherein the first container is configured to hold a maximum volume of less than 20 mL, 10 mL, 8 mL, 6 mL, 5 mL, 4 mL, 3 mL, 2 mL, or 1 mL, and/or greater than 0.1 mL, 0.2 mL, 0.5 mL, or 1 mL.

As used herein, the terms “selective” and “selectively,” when modifying an action, movement, configuration, or other activity of one or more components or characteristics of an apparatus, mean that the specific action, movement, configuration, or other activity is a direct or indirect result of user manipulation of an aspect of, or one or more components of, the apparatus.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It also is within the scope of the present disclosure that elements, components, and/or other recited subject matter that is recited as being adapted to perform a particular function may additionally or alternatively be described as being configured to perform that function, and vice versa. Similarly, subject matter that is recited as being configured to perform a particular function may additionally or alternatively be described as being operative to perform that function.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entity in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

The various disclosed elements of apparatuses and steps of methods disclosed herein are not required to all apparatuses and methods according to the present disclosure, and the present disclosure includes all novel and non-obvious combinations and subcombinations of the various elements and steps disclosed herein. Moreover, one or more of the various elements and steps disclosed herein may define independent inventive subject matter that is separate and apart from the whole of a disclosed apparatus or method. Accordingly, such inventive subject matter is not required to be associated with the specific apparatuses and methods that are expressly disclosed herein, and such inventive subject matter may find utility in apparatuses and/or methods that are not expressly disclosed herein.

It is believed that the disclosure set forth above encompasses multiple distinct inventions with independent utility. While each of these inventions has been disclosed in specific forms, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, when the disclosure, the preceding numbered paragraphs, or claims recite “a” or “a first” element or the equivalent thereof, such claims should be understood to include incorporation of one or more such elements, neither requiring nor excluding two or more such elements.

1. A method of detecting malic acid, or related species, in a liquid sample comprising:
combining a liquid sample and a reaction mixture to create a combined mixture;
incubating the combined mixture to create an incubated mixture; and
measuring a measured parameter characteristic of the incubated mixture;
wherein the reaction mixture includes a source of enzyme to transform malic acid or related species and wherein an activity concentration of the enzyme in the combined mixture is greater than 0.1 U/mL.

2. The method of claim 1, wherein the measuring includes measuring a parameter related to an amount of CO₂ generated during the incubating.

3. The method of claim 1, wherein the measuring includes measuring a pressure of a container holding the incubated mixture.

4. The method of claim 1, wherein the measuring includes measuring a pH of the incubated mixture.

5. The method of claim 1, wherein the measuring includes measuring a pH of the combined mixture before the incubating and after the incubating, and wherein the measuring includes calculating a differential pH change associated with the incubating.

6. The method of claim 1, further comprising correlating the measured parameter and the malic acid or related species concentration of the liquid sample.

7. The method of claim 1, further comprising calculating a malic acid or related species concentration of the liquid sample based upon the measured parameter.

8. The method of claim 1, further comprising determining whether a malic acid or related species concentration in the liquid sample is below, above, or approximately at a predetermined threshold based upon the measured parameter.

9. The method of claim 1, wherein the enzyme is a malolactic enzyme from any of organism that contains malolactic enzyme.

10. The method of claim 9, wherein the organism is selected from the group consisting of L. plantarum, O. oeni, S. cerevisiae, and S. pombe.

11. The method of claim 1, wherein the enzyme is a malic enzyme from any of organism that contains malic enzyme.

12. The method of claim 1, wherein the source of enzyme is a preparation of permeabilized microbes.

13. The method of claim 1, wherein the source of enzyme is a cell-free extract.

14. The method of claim 1, wherein the combining includes placing the liquid sample in a container, and further comprising sealing the container after the combining and before the incubating.

15. The method of claim 1, further comprising adjusting, before the incubating, a pH of the combined mixture to greater than about pH 5.

16. The method of claim 1, further comprising degassing the liquid sample prior to the combining, wherein the degassing includes at least one of shaking and boiling the liquid sample.

17. The method of claim 1, further comprising reducing a concentration of compounds in the liquid sample that interfere with the enzyme to transform malic acid or related species.

18. The method of claim 17, wherein the reducing includes reducing a concentration of tannins in the liquid sample and/or the combined mixture.

19. The method of claim 1, further comprising validating the measured parameter by
adding a measured amount of malic acid or related species to the incubated mixture to create a combined verification mixture;
incubating the combined verification mixture to create an incubated verification mixture; and
measuring a measured verification parameter characteristic of the incubated verification mixture;
wherein the measured verification parameter is at least one of pressure, pH, and an amount of CO₂ generated during the incubating the combined verification mixture.
